(19) United States
(12) Patent Application Publication
(10) Pub. No.: US 2006/0254788 A1
(43) Pub. Date: Nov. 16, 2006

(54) Drive and/or dose metering module with an anti-rotation stop

(76) Inventor: Eugen Bucher, Muehlethurnen (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/406,813

(22) Filed: Apr. 19, 2006

(30) Foreign Application Priority Data
Apr. 20, 2005 (DE) 10 2005 018 306.9

(51) Int. Cl. B23Q 5/00

(52) U.S. Cl. 173/178

(57) ABSTRACT

A drive and/or dose metering module for an injection device or the like, including a drive element, an output element coupled with the drive element so that a rotating movement of one of the drive element and output element about a longitudinal axis causes the other of the drive element and output element to move relative to it in the direction of the longitudinal axis, and the one which is able to effect the rotating movement has a stop which can be moved by rotation against a co-operating stop of the other and is able to prevent the rotating movement at least in one direction of rotation, wherein at least one of the stop and co-operating stop is disposed transversely to the direction of rotation.

### CROSS-REFERENCE TO RELATED APPLICATION

This application claims the benefit of German Application No. 10 2005 018 306.9, filed on Apr. 20, 2005, the entire contents of which are incorporated herein by reference.

### BACKGROUND

The present invention relates to devices for administering, delivering or dispensing substances, and methods of making and using such devices. More particularly, it relates to a drive and/or dose metering module for an injection device, in particular for an injection pen, which includes defined end stops.

Injection devices are replete in the prior art. In many such devices, a rotating movement of a drive element is converted into a longitudinal movement of a plunger rod. Some devices include a dose metering mechanism formed by the drive element and the plunger rod, wherein the mechanism has an end stop, which is able to block the movement of the plunger rod in a longitudinal direction. This end stop may be provided in the form of a stop surface disposed at a right angle to the axial direction in which the plunger rod moves. The rotating movement of the drive element is transmitted to the plunger rod via a thread, for example. In the case of small thread pitches and the specified tolerances for the plunger rod and drive element in terms of length, large variations in the terminal angle of the dose metering knob occur to a certain extent when the plunger rod is in abutment with the drive element. Depending on the torque which the user of the device applies to the drive element, the injection device can jam or break altogether.

### SUMMARY

An object of the present invention is to provide a drive and/or dose metering module for an injection device or the like which permits a defined position of the drive element in terms of rotation angle, and which enhances reliability, accuracy and durability.

In one embodiment, the present invention comprises a drive and/or dose metering module for an injection device or the like, comprising a drive element and an output element coupled with the drive element so that a rotating movement of one of the drive element and output element about a longitudinal axis causes the other of the drive element and output element to move relative to the rotating one of the drive element and output element in the direction of the longitudinal axis, the one of the drive element and output element able to effect the rotating movement having a stop which can be moved by rotation against a co-operating stop of the other of the drive element and output element and able to prevent the rotating movement at least in one direction of rotation, wherein at least one of the stop and co-operating stop is disposed transversely to the direction of rotation.

In one embodiment, a drive and/or dose metering module in accordance with the present invention comprises a drive element and an output element, which are coupled with one another such that when one of the drive element and output element effects a rotating movement, the other moves relative to it in the direction of the longitudinal axis. Whichever one of the drive element and output element is able to effect the rotating movement has a stop, which is able to prevent the rotating movement in at least one direction of rotation. The stop formed on one of the drive element and output element can be rotated against a co-operating stop provided on the other. The stop is disposed transversely to the direction of rotation. In a preferred embodiment, the drive element effects the rotating movement and the output element effects the longitudinal movement.

In one embodiment, the stop acts directly opposite the driving movement direction, as a result of which the stop force opposing the driving force does not have to pass via the element which converts the rotating movement into a longitudinal movement. For example, the rotating movement may be converted into the longitudinal movement by means of a thread or threaded drive. If using a sleeve-shaped drive element, the drive element may have an internal thread and an output element may be used in the form of a threaded rod with an external thread, so that the external and internal thread co-operate. In some embodiments, the thread pitch is small, including smaller than 45°, as a result of which the driving movement is converted into an output movement, i.e., the rotating movement is converted into the longitudinal movement. Disposing the stop surface transversely to, in some embodiments, perpendicular to, the longitudinal direction of the drive and/or dose metering module can result in a higher force acting on the stop on the one hand, and self-induced friction can occur with a small thread pitch, as a result of which the transmission mechanism and other components are subjected to a significantly higher amount of stress than is the case if the stop is disposed transversely to the direction of rotation.

In some preferred embodiments, the stop and the co-operating stop may be provided in the form of a stop surface. One stop surface sits transversely to, in some embodiments perpendicular to, the direction of rotation, in other words the normal of the stop surface points more or less in the direction of rotation. In the situation where the driving movement is converted into the output movement by means of a thread, the terms direction of rotation should also be construed a meaning the direction of a thread which, in the case of small thread pitches, deviates to some extent from the direction of rotation.

The stop and/or the co-operating stop is disposed at a radial distance apart from the longitudinal axis of the output element. The stop and/or the co-operating stop may be respectively disposed on a turn extending in the longitudinal direction, e.g., on a spiral-shaped contour on the drive element and/or the output element. Since the turn points in an end-face direction, it may extend round as far as approximately one revolution. The stop or the co-operating stop may be provided on the height offset which the turn has from its start to its end. For example, several stops or co-operating stops may be provided at a same axial position, in which case the stops or co-operating stops may be disposed transversely to, in some embodiments, perpendicular to, the direction of rotation, and the stops or co-operating stops may respectively be connected to a turn extending partially circumferentially. In some embodiments, the stops or co-operating stops are distributed uniformly around the circumference of the drive element or the output element. The product of the stop height or co-operating stop height and the number of stops or co-operating stops determines the pitch of the turn by reference to a full revolution. The turn may have a pitch which the element displaceable in the longitudinal direction covers for one revolution of the rotatable element. In some preferred embodiments, the pitch of the turn corresponds to the pitch of the thread of the threaded drive with which the drive element and the output element can be coupled. In some embodiments, the drive element has the stop or stops and the output element has the co-operating stop or stops.

If the drive element or output element is cylindrical, the turn may be formed on the cylinder barrel surface, for example on an internal or external cylinder barrel surface. In the case of a sleeve-shaped drive element or output element, the turn may also be formed by the end face of the wall of the sleeve. The turn may be a shoulder pointing essentially in the axial direction, and the turn or shoulder is bounded by an internal and an external cylinder barrel surface, and the internal and the external cylinder barrel surface are spaced at different distances with respect to the longitudinal axis. For example, the turn or shoulder may stand perpendicular on at least one of the internal and external cylinder barrel surface. The axial length by which the stop and the co-operating stop are able to come into contact may correspond to the axial distance which the element displaceable in the longitudinal direction covers with one revolution of the rotatable element.

During a rotating movement, the stop and co-operating stop can be moved towards one another. On the last revolution of the drive element before the stop comes into contact with the co-operating stop, the co-operating stop of the output element or, if one is provided, the spiral-shaped shoulder of the drive element, can assume an axial distance from the spiral-shaped shoulder of the output element that is more or less constant axially. In some preferred embodiments, the spiral-shaped contour extends around the drive element or the output element approximately in one revolution.

In a preferred embodiment, the stop is provided in the form of a displaceable cam. The displaceable cam may be provided on a resilient arm formed by the drive element. The drive element at least partially surrounds the output element. In order to join the drive element and output element, the cam, disposed on one end of the drive element, can uncover an orifice through which the output element can be inserted in the drive element. The cam may be moved approximately radially inwards, namely towards the longitudinal axis of the drive element, as a result of which the orifice is closed and the cam forms the stop for the co-operating stop of the output element. In some embodiments, a locking element may be secured to the drive element, which is able to move the cam more or less radially inwards when it is or becomes secured to the drive element. In some embodiments, the locking element may be screwed onto the drive element by means of a thread. The resiliently mounted arm may have the shape of the spiral-shaped shoulder described above. The arm forms a spiral-shaped shoulder once the cam has been moved inwards.

In one preferred embodiment, the output element has two co-operating stops disposed transversely to the direction of rotation, and of the two co-operating stops, the first is able to prevent a rotating movement in the first direction of rotation and the second is able to prevent a rotating movement in the second direction of rotation of the drive element. The co-operating stops may point in different directions of rotation. For example, the spiral-shaped shoulders co-operating with the respective co-operating stops may point in different axial directions. With an output element of this design, a drive element adapted to it may have a stop disposed on one shoulder and a stop formed by the displaceable cam which can be rotated each against a co-operating stop. The stops point in different directions of rotation. The different directions of rotation of the spiral-shaped shoulders assigned to the respective stops may point in different axial directions.

The output element can be moved by rotating the drive element between the positions in which a stop is rotated on a co-operating stop. The output element can be moved by the drive element, e.g. in several steps, in the direction of the longitudinal axis, in which case the drive element may also serve as a dose metering element. The invention may also be used in a so-called automatic injector in which the drive element is able to drive the output element in an axial forward-drive direction, e.g., for a single movement.

A device, and/or components and/or modules thereof, in accordance with the present invention may be made completely or partially from plastic, such as 40% plastic reinforced with carbon fibre, although other suitable materials may be used as well, in whole or in part.

### BRIEF DESCRIPTION OF THE DRAWINGS

FIG. 1 a longitudinal section of one embodiment of a device in accordance with the present invention with a cam not pushed in,

FIG. 2 is a longitudinal section of the device illustrated in FIG. 1 with a cam pushed in by a screwed on locking element,

FIG. 3 shows a portion of one embodiment of a drive element incorporating a stop,

FIG. 4 shows an embodiment of an output element, with a rear co-operating stop,

FIG. 5 shows an embodiment of an output element, with a front co-operating stop, and

FIG. 6 is a perspective view of the drive element illustrated along section A-A of FIG. 1.

### DETAILED DESCRIPTION

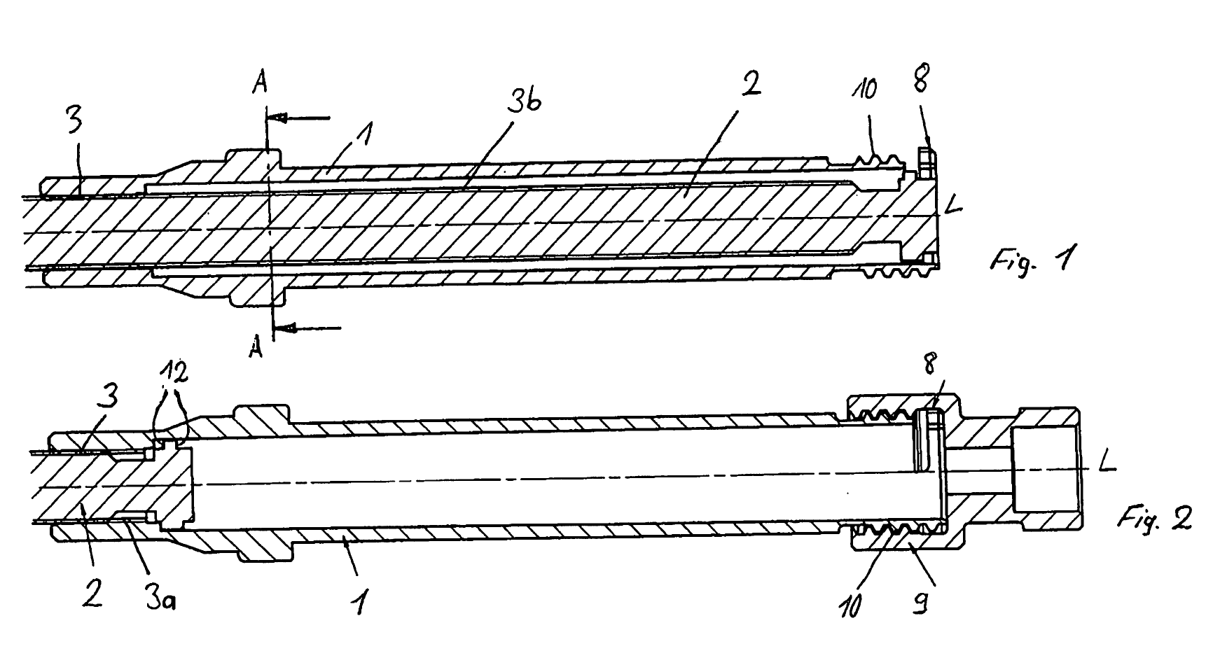

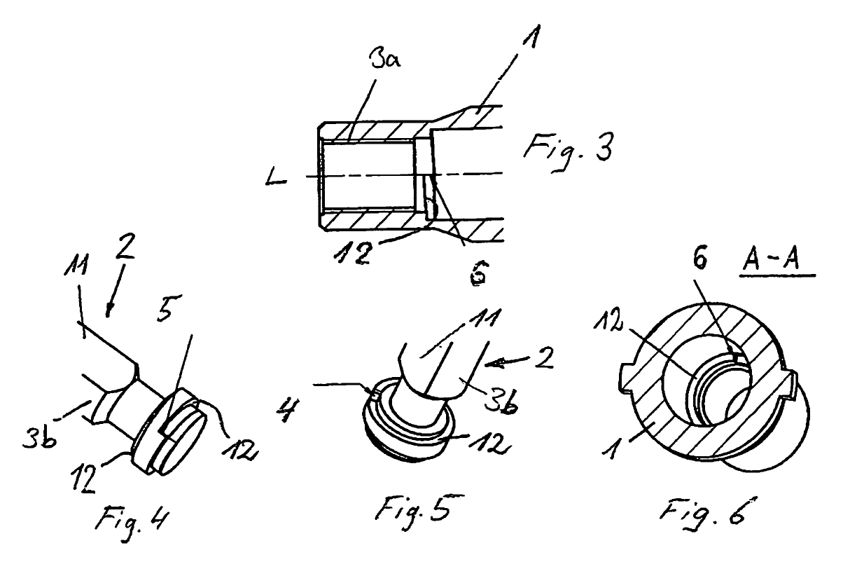

FIG. 1 illustrates an embodiment of a drive and/or dose metering module in accordance with one embodiment of the present invention. The module has a drive element 1, which has a threaded nut portion. The drive element 1 is sleeve-shaped and surrounds an output element 2. The drive element 1 has an internal thread 3a and the output element 2 has an external thread 3b, as illustrated in FIGS. 3 and 4. The mutually engaging threads 3a, 3b form a threaded drive 3, which converts a rotating movement of the drive element 1 into an axial movement of the output element 2 along the longitudinal axis L.

The drive element 1 may be mounted by means of a housing (not illustrated), so that it can rotate and is axially stationary, for example. The output element 2 may be mounted by the housing (not illustrated), so that it is axially displaceable and so that it can not rotate. To this end, the output element 2 has at least one guide surface 11, in some embodiments, two guide surfaces 11, on the circumference incorporating the thread 3b (FIG. 4). The guide surfaces 11 are flat regions with no thread 3b and, in conjunction with a complementary element adapted accordingly, prevent the output element 2 from rotating.

As illustrated in FIGS. 1 and 2, the drive element 1 has an orifice which can be at least partially closed by means of a cam 8. The cam 8 is formed on a radially resilient arm of the drive element 1. The drive element 1 also has a thread 10, on which the locking element 9 in the form of a threaded cap can be screwed. When the locking element 9 is not screwed onto the drive element 1, the surface of the arm pointing radially outwards assumes a position at a bigger radial distance from the longitudinal axis L than the core radius of the thread 10. When the locking element 9 is screwed on, the cam 8 is moved by the thread tips of the locking element 9 approximately radially inwards, namely transversely to the longitudinal axis L. When the locking element 9 is screwed on, the cam 8 forms a stop, in particular an anti-rotation stop for the output element 2, in particular for a co-operating stop 5 of the output element 2.

At its end pointing in the direction of the cam 8, the output element 2 has a head, the diameter of which is bigger than the thread diameter. An undercut is formed between the head and thread 3b. As illustrated in FIGS. 4 and 5, the head has a rear co-operating stop 5, which sits in an anti-rotation abutment with the cam 8 when the drive element 1 is moved in rotation about the output element 2 once the output element 2 has moved as far as the cam 8 due to the rotating movement of the drive element 1. Any further rotating movement of the drive element 1 about the output element 2 is then prevented, at least in one direction. The rear co-operating stop 5 provided on the output element 2 comprises a surface disposed transversely with respect to the direction of rotation or in the direction of the longitudinal axis L. The rotating movement of the drive element 1 is directly blocked at least in one direction as a result because the cam 8 serving as a stop is rotated or pivoted against the co-operating stop 5 of the output element 2. The rear co-operating stop 5 is formed on a spiral-shaped contour 12 extending around a revolution along the longitudinal axis L. A purpose of the spiral-shaped contour 12 is to ensure that the output element 2 does not move into any longitudinal abutment with the cam 8 during the last revolution of the drive element 1 before the cam 8 is rotated into contact with the rear anti-rotation stop 5.

A front co-operating stop 4 is also provided on the head of the output element 2, an anti-rotation stop, which is generally of the same design as the rear co-operating stop 5. The front co-operating stop 4 is formed on a spiral-shaped contour 12 extending around one revolution. The front stop 6 can be rotated against the front co-operating stop 4 in order to prevent the drive element 1 from rotating about the output element 2 in at least one direction. The co-operating stop 6 is formed by a shoulder of the drive element 1 extending radially inwardly, as may be seen from FIGS. 3 and 6. A spiral-shaped contour 12 extending around one revolution is also provided on the shoulder of the drive element 1 extending radially inwardly, which, in conjunction with the contour 12 assigned to the front co-operating stop 4, prevents the output element 2 from moving into an axial contact with the drive element 1 before the front stop 6 has been rotated or pivoted against the front anti-rotation stop 4. As a result of the co-operating stops, which are disposed transversely to, in some embodiments perpendicular to, the direction of rotation, the output element 2 can be moved relative to the drive element 1 between the positions defining exact end angles in which the front stop 6 comes into contact with the co-operating stop 4 or the cam 8 comes into contact with the rear co-operating stop 5.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A drive and/or dose metering module for an injection device, in particular for an injection pen, comprising:
a) a drive element,
b) an output element, which is coupled with the drive element so that a rotating movement of one of the drive element and output element about a longitudinal axis causes the other of the drive element and output element to move relative to it in the direction of the longitudinal axis,
c) and the one which is able to effect the rotating movement has a stop which can be moved by rotation against a co-operating stop of the other and is able to prevent the rotating movement at least in one direction of rotation,
characterised in that
d) at least one of the stop and co-operating stop is disposed transversely to the direction of rotation.

2. The drive and/or dose metering module as claimed in claim 1, wherein the drive element is able to effect the rotating movement and the output element is able to effect the longitudinal movement.

3. The drive and/or dose metering module as claimed in claim 2, wherein the drive element has the stop which is able to prevent the rotating movement in at least one direction of rotation the drive element.

4. The drive and/or dose metering module as claimed in claim 3, wherein the stop is disposed radially at a distance apart from the longitudinal axis of the output element.

5. The drive and/or dose metering module, as claimed in claim 4, wherein the stop is disposed on a spiral-shaped contour.

6. The drive and/or dose metering module as claimed in claim 1, wherein the drive element and output element are coupled with a threaded drive.

7. The drive and/or dose metering module as claimed in claim 1, wherein the output element has two co-operating stops disposed transversely to the direction of rotation, and of the two stops, a first is able to prevent a rotating movement of the drive element in a first direction of rotation and a second is able to prevent a rotating movement of the drive element in a second direction of rotation.

8. The drive and/or dose metering module as claimed in claim 1, wherein the drive element has a displaceable cam, which is provided as a stop for a co-operating stop of the output element.

9. The drive and/or dose metering module as claimed in claim 8, wherein a locking element can be secured to the drive element, which is able to move the cam approximately radially inwards when it is secured to the drive element.

10. The drive and/or dose metering module as claimed in claim 9, wherein the locking element can be screwed onto the drive element by means of a thread.

11. The drive and/or dose metering module as claimed in claim 10, wherein at least one of the drive element, output element and locking element is made from plastic.

12. A drive and/or dose metering module for an injection device or the like, comprising:
a drive element;
an output element coupled with the drive element so that a rotating movement of one of the drive element and output element about a longitudinal axis causes the other of the drive element and output element to move relative to the rotating one of the drive element and output element in the direction of the longitudinal axis, the one of the drive element and output element able to effect the rotating movement having a stop which can be moved by rotation against a co-operating stop of the other of the drive element and output element and able to prevent the rotating movement at least in one direction of rotation, wherein at least one of the stop and co-operating stop is disposed transversely to the direction of rotation.
